(19) United States
(12) Patent Application Publication
(10) Pub. No.: US 2004/0234933 A1
(43) Pub. Date: Nov. 25, 2004

(54) Medical procedure training system

(76) Inventors: Steven L. Dawson, Carlisle, MA (US); Mark Peter Ottensmeyer, Cambridge, MA (US); Stephane M. Cotin, Belmont, MA (US); Paul Francis Newmann, Boston, MA (US)

Correspondence Address: Paul D Durkee, Daly Crowley & Mofford, Suite 101, 275 Turnpike Street, Canton, MA, 02021-2310, US

(21) Appl. No.: 10/488,415

(22) PCT Filed: Sep. 9, 2002
(86) PCT No.: PCT/US02/28593

Related U.S. Application Data
(60) Provisional application No. 60/318,033, filed on Sep. 7, 2001.

(51) Int. Cl. G09B 23/28; A61B 5/05

(52) U.S. Cl. 434/262; 600/424

(57) ABSTRACT

A medical procedure training system includes a mannequin having internal and external anatomical characteristics, such as body contour and organs, derived from medical imaging of an actual patient. The system can includes an instrument tracking system for monitoring the position of an instrument in relation to the mannequin anatomical components, which can be registered in relation to a reference point.

### STATEMENT REGARDING FEDERALLY SPONSORED RESEARCH

The Government may have certain rights in the invention pursuant to Department of Defense grant DAMD 17-99-2-9001, as amended with funds from Research Area Directorate II/Combat Casualty Care.

### FIELD OF THE INVENTION

The present invention relates generally to surgical training and, more particularly, to devices and systems for providing realistic training in surgical procedures.

### BACKGROUND OF THE INVENTION

As is known in the art, the quality of medical training in surgical procedures is a factor in the success rate for actual procedures. The more realistic the training that is received, the more prepared medical personnel will be under actual conditions. A variety of known devices have been developed to train medical personnel for surgical procedures including mannequins having one or more parts generally corresponding to anatomical features. Such devices can be used to provide some degree of training for diagnosis and/or treatment of a trauma. However, these devices typically focus on visual anatomical similarity. That is, the haptic sensations received during a training procedure will be quite different than that experienced during an actual procedure. Exemplary surgical training devices and systems are available from Limbs and Things Ltd of Bristol England (www.limbsandthings.com).

The military need for effective training in acute penetrating trauma is well known. For example, death can unnecessarily result from unrecognized or untreated but potentially survivable penetrating injury. Tension pneumothorax, for example, is the second leading cause of battlefield death in casualties that survive an initial injury. However, known surgical training techniques for such procedures are limited to training procedures on animals, unrealistic models, and computer simulations or virtual procedures. Such techniques have various shortcomings that are well known to those who have performed actual procedures.

It would, therefore, be desirable to overcome the aforesaid and other disadvantages.

### SUMMARY OF THE INVENTION

The present invention provides a surgical training system including a mannequin having anatomical characteristic derived from an actual patient. With this arrangement, medical training procedures can be performed on a mannequin having realistic internal and external features. While the invention is primarily shown and described in conjunction with a human mannequin for chest trauma treatment training, it is understood that the invention is applicable to surgical training in general for which a wide range of surgical procedures will be performed.

In one aspect of the invention, a human male was medically imaged using computed tomography to generate a set of relatively high quality images of the subject. In one particular embodiment, images of the chest and upper abdomen were obtained. The image set was segmented using a suitable three dimensional software application. An exemplary segmentation provided discrete anatomic components including lungs, mediastinum, ribs, skin, and certain abdominal organs. The segmented dataset was transformed into various subsets of three dimensional models for the anatomic components using a known software application. Molds for the anatomic components were then generated from the three dimensional models. The molds were then used to cast the mannequin components, which were then assembled to provide an anatomically accurate model of the patient.

In another aspect of the invention, the medical procedure training system includes an instrument tracking module for tracking one or more instruments in relation to the mannequin. Each instrument, such as a chest tube, includes a sensor that provides a position and rotation of the instrument at any given time in response to a transmitted signal. The emitter module can be affixed to the mannequin at a known location so that position and orientation of a given instrument is known in relation to the mannequin based upon the signal return. The same data models used to fabricate the mannequin are used as a reference model for the tracking module, thus ensuring consistency between the physical and virtual representations of the anatomy.

In another aspect of the invention, the medical procedure training system includes a special effects module to enhance the realism of a training procedure. In one embodiment, the special effects module, in combination with the instrument tracking module, can selectively provide blood and air release based upon a position of a tracked instrument. For example, the special effects module can generate synthetic blood flow when a chest tube is placed into a simulated hemothorax. Similarly, computer-generated sounds can be produced to mimic the “gush of air” associated with the treatment of a tension pneumothorax. Air release, sounds, instructions, and the like, can be generated by the special effects module.

In a further aspect of the invention, the medical procedure training system can include a module for evaluating trainee performance based upon the position of various tracked instruments for given procedures. The tracking sensors measure the position and orientation of instruments, such as the chest tube and decompression needle, with respect to the mannequin. Collision detection provides real-time feedback about potential contacts with internal organs, thereby minimizing instructor supervision. Collision detection is based on virtual representations of thoracic organs that match and have been registered with the models in the mannequin. Sensor position and orientation data is used to assess chest tube or needle placement inside the chest cavity. This information is computed in real-time and played back upon completion of the procedure. Upon trainee error, the anatomy and position of the instruments are displayed on the monitor.

In another aspect of the invention, the present invention provides a surgical training system including a portal having an anatomically analogous structure generating realistic haptic feedback during surgical training. With this arrangement, the level of surgical training is enhanced so that trainees are well prepared for actual surgical procedures. While the invention is primarily shown and described in conjunction with chest portals and treating penetrating trauma injuries, it is understood that the invention is applicable to surgical training portals in general at various bodily locations in which realistic haptic feedback is desirable.

In one aspect of the invention, a lateral chest portal includes a support structure to which a plurality of members corresponding to ribs are secured. A first material corresponding to intercostal muscle is secured to the rib members and a first layer corresponding to a pleura layer is disposed adjacent to an interior side of the intercostal material. In one particular embodiment, the ribs are embedded in the intercostals muscle material. A second layer corresponding to subcutaneous fat is located adjacent the exterior side of the intercostal material and a third layer corresponding to skin is disposed adjacent the subcutaneous fat to form an outermost layer. In one embodiment, the lateral chest portal is suitable for simulating chest tube insertion for pneumothorax, hemothorax, and tension pneumothorax injury treatment.

In another aspect of the invention, a surgical training system includes a torso shell having an aperture adapted for receiving the lateral chest portal. The portal can be removably inserted into the torso shell aperture to enhance the training experience. A flexible outer layer can be disposed over the shell for a more realistic look and feel for the torso.

In a further aspect of the invention, a surgical training system includes a torso shell having apertures corresponding to a location at which tension pneumothorax is treated with a surgical dart. The torso shell is covered with a outer layer incorporating skin and subcutaneous fat and/or muscle. In one embodiment, the outer layer comprises the layers of the chest external to the ribs in one generally continuous structure. The skin and muscle layers provide haptic feedback that emulates the feel of inserting a surgical dart into the chest cavity between upper ribs of a patient.

### BRIEF DESCRIPTION OF THE DRAWINGS

The invention will be more fully understood from the following detailed description taken in conjunction with the accompanying drawings, in which:

FIG. 1 is a pictorial representation of a surgical training system in accordance with the present invention;

FIG. 2 is a pictorial representation of a CAD model of a mannequin (based on an imaged human male torso) that can be used to fabricate the mannequin of FIG. 1;

FIG. 3 is a pictorial representation of a torso shell that can form a part of the mannequin of FIG. 1;

FIG. 4A is a pictorial representation of collision detection for a training system in accordance with the present invention;

FIG. 4B is a pictorial representation showing further details of 3D collision detection;

FIG. 5 is a pictorial representation of certain instruments that can be tracked during medical training procedures in accordance with the present invention;

FIG. 5A is a schematic depiction of a chest tube including a sensor by which a position of the chest tube in relation to the mannequin can be tracked in accordance with the present invention;

FIG. 5B is a pictorial representation of a surgical dart and syringe having a removable sensor in accordance with the present invention;

FIG. 6 is a pictorial representation of a computer and special effects module that form part of the medical procedure training system of the present invention;

FIG. 7 is a schematic diagram of an exemplary implementation of the special effects module of FIG. 6;

FIG. 7A is a schematic depiction of audio sources for providing sound effects for a medical procedure training system in accordance with the present invention;

FIG. 8 is a schematic depiction of a system software architecture showing an augmented reality interface and user interface that can form a part of the medical procedure training system in accordance with the present invention;

FIG. 9 is a pictorial representation of a medical procedure training system having a display secured to a litter in accordance with the present invention;

FIG. 10 is a schematic representation of the medical training system in accordance with the present invention;

FIG. 11 is a partially exploded pictorial representation of a surgical training system including a portal in accordance with the present invention;

FIG. 11A is a pictorial representation of the surgical system of FIG. 11 showing a portal in accordance with the present invention secured to a torso shell, which can form a part of the surgical training system;

FIG. 11B is a further pictorial representation of the surgical training system of FIG. 11 further showing a soft outer layer over the torso shell;

FIG. 12 is a pictorial skeletal representation of the real anatomy on which the surgical training system of FIG. 11 is modeled;

FIG. 12A is a cross-sectional view of the portal shown in FIG. 12 taken along line A-A along with supporting margins of a torso shell;

FIG. 12B is a pictorial representation of a exemplary engagement mechanism for securing the portal of FIG. 12 to a torso shell in accordance with the present invention;

FIG. 13 is a pictorial representation of further surgical training system for tension pneumothorax in accordance with the present invention; and

FIG. 13A is a cross-sectional view of the tension pneumothorax portal of FIG. 13; and

FIG. 13B is a cross-sectional view of an alternative embodiment of the tension pneumothorax portal of FIG. 13.

### DETAILED DESCRIPTION OF THE INVENTION

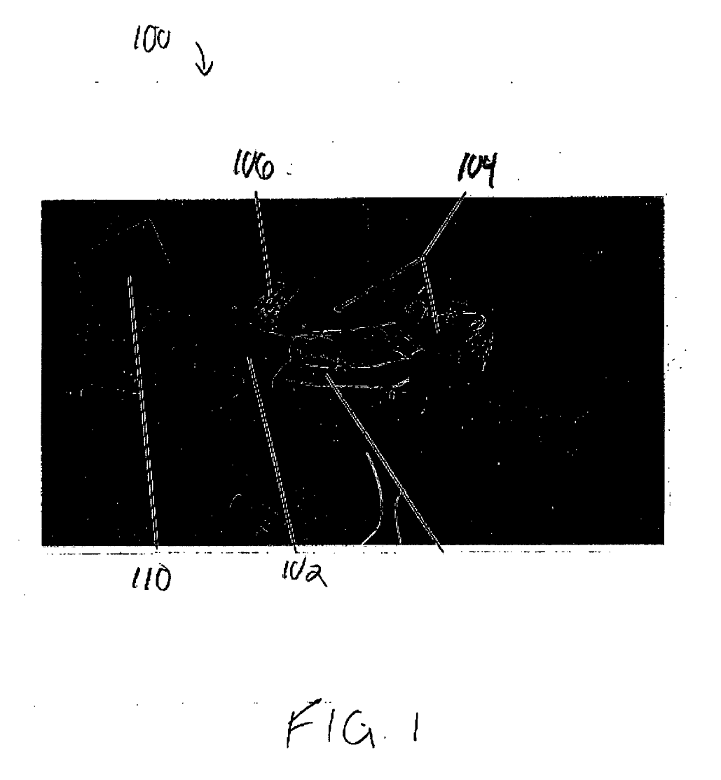

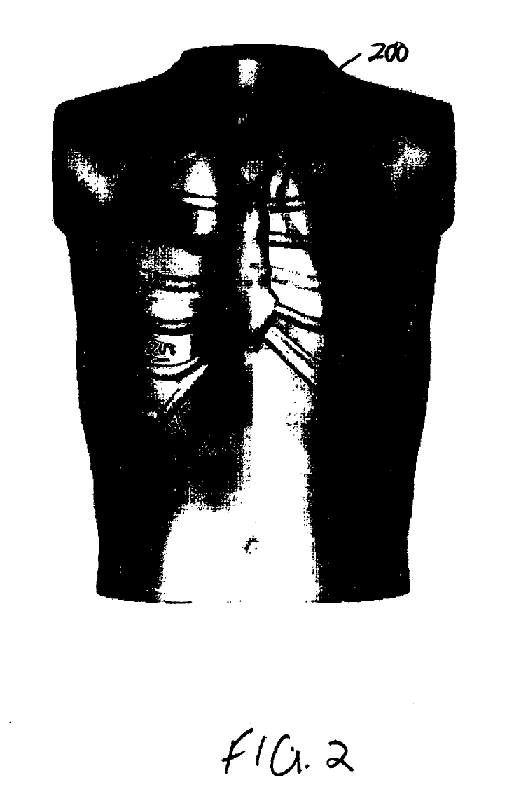

FIG. 1 shows an exemplary medical training system 100 having a mannequin 102 with anatomical characteristics derived from an actual human male. In general, Computed Tomography (CT) images of the patient were used to model and construct the mannequin 102. The system 100 includes a controller system 104, such as a laptop computer, for monitoring and controlling the overall training system. A special effects module 106 is coupled to the controller 104 for enhancing the realism of the training procedure by producing sound, simulated blood, air pressure/release and the like. The controller 104 can include an instrument tracking system 108 that operates in conjunction with the special effects module 106 so that the appropriate effects are activated in response to the location and movement of the instruments during a training procedure. The system 100 can further include a display system 110, such as a touch panel display, for interaction with the user.

In one aspect of the invention, the mannequin 102 has the anatomical characteristics of an adult male of physique approximating that of a typical male soldier. The patient was scanned using a CT system and approximately 500 slices extending from the neck through the upper abdomen were obtained. It is understood, however, that more and fewer images can be taken depending upon the requirements of a particular application. It is further understood that other types of imaging systems can be used without departing from the invention.

In one embodiment, the DICOM (Digital Imaging and Communications in Medicine) standard format data was imported into an image processing software called 3D-doctor, which is available from Able Software Company of Lexington, Massachusetts. For the purposes of accurately segmenting the relevant anatomy, a semi-automatic segmentation was performed: after an initial fully-automated segmentation computed by the image processing software, the boundaries of each organ were manually adjusted (by a medical expert) using a specific interface provided by the same software. Upon completion of the segmentation, a set of three-dimensional models of each organ was created from the set of two-dimensional boundaries after careful smoothing of the boundaries was applied and each organ boundary was labeled with a unique identifier. The segmented dataset was exported from 3D-Doctor as STL files (standard format for stereolithography process) and then converted into a set of three-dimension CAD files using a combination of software, mainly a 3D modeling package, Rhinoceros 3D by Robert McNeel & Associates, and a three dimensional CAD software application, such as Solidworks software by Solidworks Corporation of Concord, Massachusetts. The models are then modified such that a mannequin, with features including, the hard shell between the palpable ribs, the removable mediastinum, lungs and diaphragm, and other components, can be manufactured. The 3D CAD file was used to generate rapid prototype models for use in creating molds for fabricating the mannequin parts.

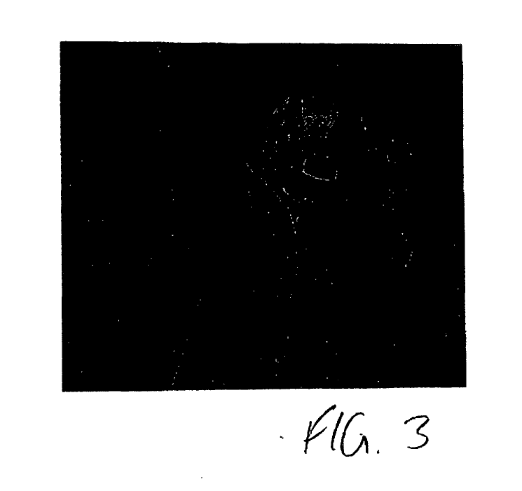

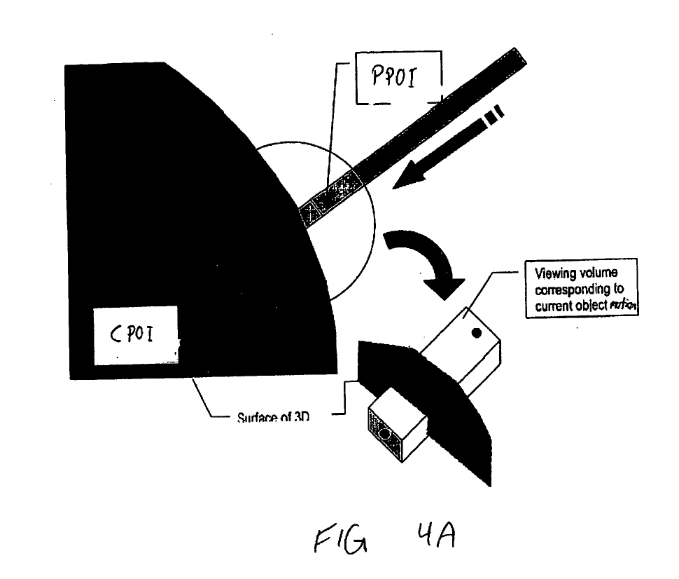

In an exemplary embodiment shown in FIG. 2, segmented anatomical components include the outer surface for the skin 200, the rib cage 202, the mediastinum 204, the lungs 206, and the diaphragm 208. As shown in FIG. 3, the mannequin can include a relatively rigid torso shell 250 around which a skin-like outer layer can be overlaid. Internal organs can be contained within the torso shell 250.

In one particular embodiment, the anatomical components are fabricated and assembled by Limbs and Things of Bristol, England. Unless otherwise specified, part numbers refer to Limbs and Things part numbers. The torso shell is formed from semi-rigid polyurethane, which can be provided as Chest Drain Rib Material 1.1. The torso shell should be sufficiently strong to withstand the pressures expected during various surgical procedures to treat chest trauma, for example, such as hemothorax, pneumothorax and tension pneumothorax. The skin 200, which should be elastically deformable and “feel” like actual skin, can be provided as Chest Drain Skin material version 1.1. The lungs 206 can be provided as Chest Drain Skin material version 1.1, which is a polyurethane foam material. The mediastinium 204 can also be provided as Chest Drain Skin material version 1.1. The diaphragm 208 should be elastically deformable and can be provided as Chest Drain Diaphragm material version 1.1.

As described more fully below, in addition to enabling the fabrication of realistic anatomical components, the CAD models are also used to create virtual representations of the anatomy to be used in a real-time collision detection algorithm. The purpose of the collision detection module is to provide immediate feedback to the trainee according to the motion of the tracked instruments. The feedback can include sensory information related to the normal course of the procedure or information regarding a mistake that has been detected (e.g. a lung has been punctured). By creating the virtual representation of the anatomy from the CAD models used to fabricate the mannequin parts, a one-to-one correspondence can be defined between the virtual models and the mannequin. This correspondence is defined as a rigid transformation (3 degrees of translation, 3 degrees of rotation) that maps the position of a tracking sensor into the virtual space. This transformation is defined as the relative translation and rotation of the reference frame of the CAD models and the reference frame of the tracking system. In one embodiment, an electromagnetic field emitter of the instrument tracking system is rigidly attached to the torso shell so that the rigid transformation between the mannequin and virtual anatomy is maintained even when the mannequin is moved and without requiring any calibration when the system is started.

The collision detection module detects, in real-time, contacts between a tracked instrument and a virtual anatomic structure. In an exemplary embodiment, the algorithm is based on the OpenGL software library and takes advantage of the 3D graphics hardware of the computer to perform the various operations involved in detecting collisions. A collision is defined as an intersection between a so-called parallelepiped and a set of triangles defining the surface of the 3D model. The parallelepiped section is a function of the size of the tracked instrument (e.g., radius of the needle, radius of the chest tube) and its length corresponds to the distance between to successive locations of the tracked instrument. The speed of the algorithm depends on the graphics hardware and tracking system update rate.

The well known OpenGL Application Programming Interface (API) provides a mechanism for picking objects in a 3D scene using the mouse, i.e., for identifying what (part of an) object is located “below” the mouse pointer (a 2D point). As known to one of ordinary skill in the art, OpenGL is a cross-platform standard for 3D rendering and 3D hardware acceleration. The collision detection module relies on this mechanism and extends it to the case of a 3D moving point.

Detecting a collision between two three-dimensional objects includes testing if the volume of the first object (e.g. an instrument) intersects the second object (e.g. an organ). This process has similarities with a scene visualization process where the programmer specifies a viewing volume (or frustum) characterized by the location, orientation and projection of a camera. One part of the process includes rendering only the part of the scene contained in the viewing volume. Since specialized graphics hardware performs this very efficiently, the real-time collision detection algorithm does not increase the load of the CPU. In general, a viewing volume is specified that corresponds to the volume covered by a three-dimensional point between two consecutive time steps.

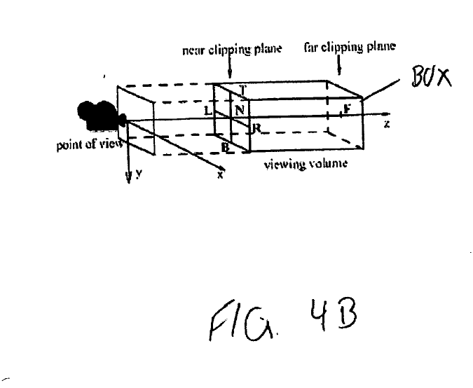

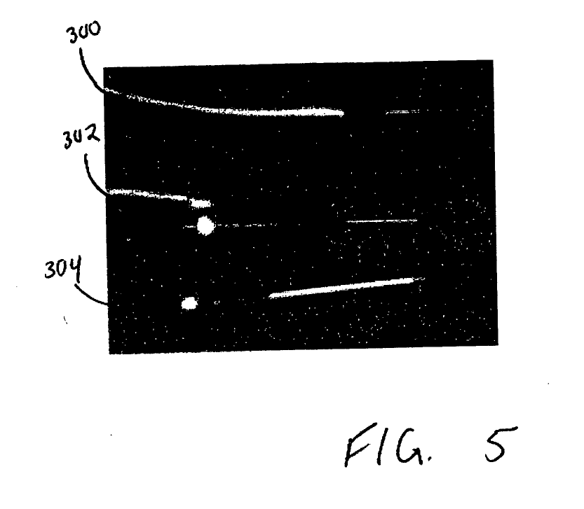

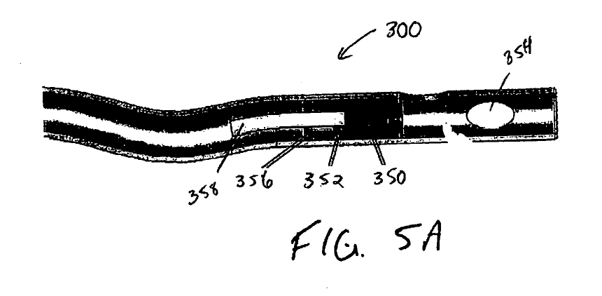

As shown in FIG. 4A, a point of interest (POI) is typically defined on a tracked instrument and can correspond, for instance, to the tip of a needle. A point of interest can have a current location CPOI, which may have moved from a previous location PPOI. The location of the point on the moving object depends on the shape and purpose of the instrument. The number of points of interest can be greater than one. The viewing volume is defined as a parallelepiped, thus corresponding to an orthographic camera as shown, which is supported by the OpenGL library. By requesting the graphics hardware to render the scene (e.g. set of anatomic structures) relatively to this “camera”, it can be known whether or not a collision has occurred: if nothing is visible, there is no collision; otherwise the system can obtain meaningful information regarding the (part of the) object that intersects with the trajectory of the instrument.

FIG. 4B shows an exemplary depiction of an OpenGL orthographic camera. The viewing volume is a parallelepiped BOX characterized by the distances to the far and near clipping planes and by the two intervals [left, right] and [top, bottom] which define their section in the near clipping plane.

The following description illustrates an exemplary sequence of the steps for detecting which objects intersect with a 3D moving point (i.e. viewing volume):

1. Get current (Pₜ) and previous (Pₜ₋₁) coordinates of the moving point

2. Define viewing volume/orthographic camera based on (Pₜ) and (Pₜ₋₁)

3. Render the scene, using primitives relevant to the collision detection

4. Identify the primitives (if any) which were rendered by the orthographic camera

5. Process collision information (i.e. if contact detected with lung, stop tracking instrument, and show error message.).

In order to identify the rendered objects using the exemplary OpenGL API, all relevant objects in the scene are named (i.e., given a unique identifier). The OpenGL API allows giving names to primitives, or sets of primitives (objects). The OpenGL API provides a special rendering mode, called selection mode, that does not render the objects but instead store the names of the objects (plus additional information) in an array. Using the OpenGL terminology, each name stored in this array is called a hit. By parsing the hit records it is possible to identify what object (or primitive) has been collided.

In an exemplary algorithm, shown below, to implement collision detection in accordance with the present invention, x, y, and z correspond to the coordinates of the current point of interested on the tracked instrument and xp, yp, and zp are the coordinates of the previous point.

// Switch rendering mode to selection mode
glRenderMode(GL_SELECT);
P.Set(xp, yp, zp); // previous position defined in eye coordinates
Po.Set(x, y, z); // current position defined in eye coordinates
xp = x;
yp = y;
zp = z;
// compute distance between far and near clippting planes
PoP = P − Po;
L = PoP.norm( );
// instrument section expressed in eye coordinates
s = instrument.GetSection( );
// Switch to modelview matrix mode and save the matrix
glMatrixMode(GL_MODELVIEW);
glPushMatrix( );
glLoadIdentity( );
// Move the camera to set eye at Po and looking at P
gluLookAt(x, y, z, P.x, P.y, P.z, 0.0, 1.0, 0.0);
// Switch to projection and save the matrix
glMatrixMode(GL_PROJECTION);
glPushMatrix( );
glLoadIdentity( );
// Establish new clipping volume
glOrtho(−s, s, −s, s, 0, L);
// Draw the scene with ‘names’ associated with geometric primitives
DisplayAnatomy(GL_SELECT);
// Collect the hits
hits = glRenderMode(GL_RENDER);
// If a hit occured, process the info return by OpenGL
if (hits >= 1)

objectID = processHits(hits, selectBuff, x, y, z);

// Restore the modelview matrix
glMatrixMode(GL_MODELVIEW);
glPopMatrix( );
// Restore the projection matrix
glMatrixMode(GL_PROJECTION);
glPopMatrix( );

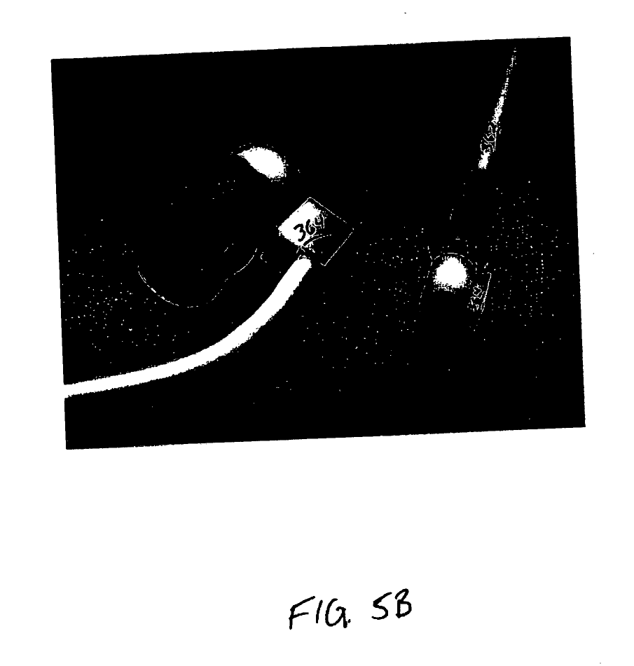

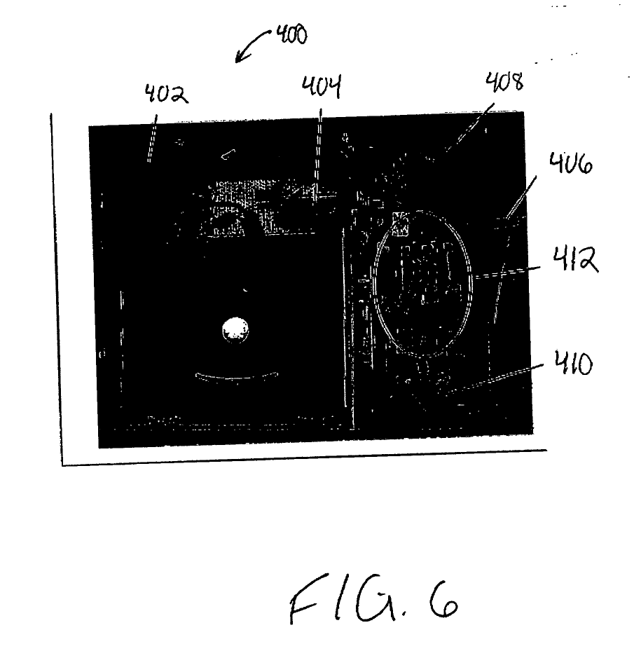

It is understood that a variety of instruments can be used and/or tracked for particular surgical training procedures. Exemplary instruments used during the course of treating conditions simulated by the mannequin include untracked tools (e.g., titanium hemostat/Kelly clamp, needle and suture, disinfectant, gauze) and tracked instruments (e.g., chest tube, decompression needle/chest dart, anesthetic syringe). Illustrative trackable instruments are shown in FIG. 5 as a chest tube 300, an anesthetic syringe 302 and a chest dart 304. Each trackable instrument includes at least one sensor.

FIG. 5A shows an exemplary chest tube 300 having a sensor 350 for enabling positional tracking by the tracking module 108 (FIG. 1). In one particular embodiment, a pulsed DC magnetic sensor system is used, such as miniBird sensors from Ascension Technologies of Burlington, Vt. The sensors are positioned on the instruments and an associated application in the tracking system tracks the instruments in response to a transmitted signal. Sensor tracking is well known to one of ordinary skill in the art. The trackable test tube 300 can also include a sensor housing 352 for containing the sensor in a fixed position.

In one embodiment, a 5 mm miniBird sensor 350 is mounted in a cylindrical housing 352, which is press fit into the chest tube. The chest tube is non-standard in that only two side holes 354 near the distal end of the tube are included. In a conventional chest tube there are typically 4-6 holes to permit drainage through the tube at locations other than the tip. The cylindrical sensor housing 352 is mounted just proximal from the proximal side hole 354. The cylinder is crafted such that a threaded rod can be mated with a socket in the housing, permitting it to be drawn out for replacement, and reinserted to the proper depth. A washer-shaped soft rubber gasket 356 is placed around the sensor cable 358, proximal from the sensor housing. This gasket prevents the artificial (or simulated) blood, which is described below, from exiting the distal end of the tube so as to force the artificial (or simulated) blood to drain through the (normal) proximal end.

As shown in FIG. 5B, a trackable syringe 360 and chest dart 362 include an attachment mechanism 364, such as a quick-change dovetail fixture, that permits the attachment, alignment and exchange of a second miniBird sensor between each of these instruments. A fixture is bonded to the sensor itself, with a dovetail socket. It is pushed manually onto the dovetail protrusion attached to each of the syringe and chest dart. As discussed below, the transformation vector between the mounted miniBird sensor and the tips of each of the syringe and chest dart is known, and is reliably reproduced because of the relatively tight fit between the fixtures on the sensor and instruments.

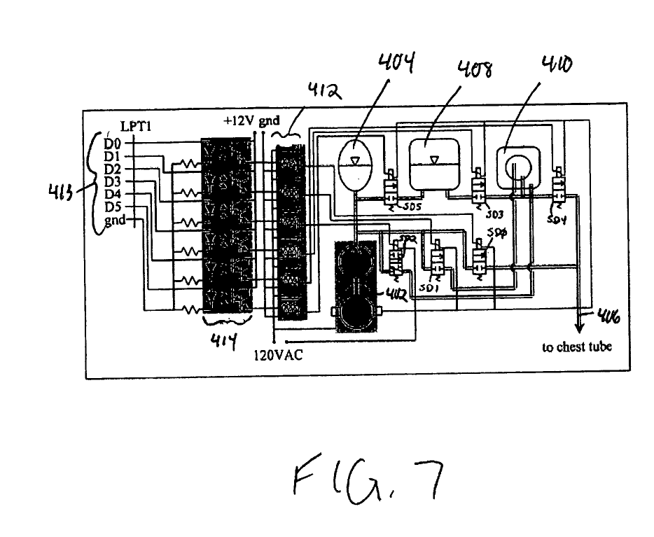

FIG. 6 shows an exemplary special effects module 400, which can correspond to the special effects module 108 of FIG. 1. The special effects module 400 provides realistic feedback during surgical training procedures, such as from the chest tube tracked instrument. It is understood that the special effects module 400 can interact with a collision detection module described below so that instrument location can generate the various special effects. On successful placement of the tube into the chest cavity for a simulated hemo- or hemo-pneumothorax, for example, artificial (or simulated) blood is driven by the module 400 through the chest tube such that it recreates the experience of performing the procedure on a patient. Similarly, if a pneumothorax is simulated, air is emitted from the tube, as if air within the pleural space is released through the chest tube.

The special effects module 400 includes an air compressor 402, an air accumulator 404 and air/fluid outlet 406 for providing pressurized air to the chest tube (see FIG. 4). The module further includes a blood reservoir 408 and a measured fluid container 410. A series of solenoid valves 412 are activated to generate blood flow and air discharge, as described below.

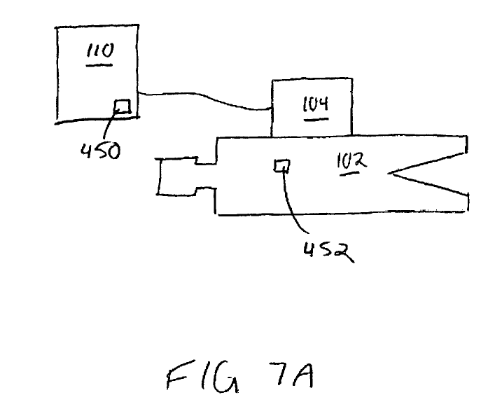

FIG. 7 shows an exemplary schematic for the electro-pneumo-hydraulic system components of the module of FIG. 6. In an exemplary embodiment, the special effects module 400 includes a connector 413 for coupling to a parallel port of the lap top computer 104 (FIG. 1) to control the solenoid valves 412 via opto-isolators 414, which permit transfer of “blood” from the reservoir 408 to the measured chamber 410 and release of fluid (air and/or “blood”) from the system. The electronics can be powered by 110/120VAC power and 12VDC supplied by an onboard transformer.

As shown in FIG. 7A, in an exemplary embodiment audio feedback during training procedures can be provided for verbal feedback and realistic effects. A first speaker 450 can be provided as a speaker coupled to a display, such as the touch screen 110 of FIG. 1. The first speaker 450 can produce the audio from synthesized speech as part of the user interface, as well as the cue sounds including the heart-rate monitor. A second speaker 452 can be provided as a non-ferrous, flat panel piezo-electric loudspeaker, for example, mounted within the mannequin torso 102 (FIG. 1). This type of speaker minimizes interference with the tracking system. The second speaker 452 produces, for example, an audio cue for the insertion of the chest dart in the form of the sound of air hissing out of the needle. In addition, for environments with significant interfering noise, additional amplifiers and loudspeakers (such as desktop computer speakers) may be added to the system for additional volume.

In another aspect of the invention, the surgical training system can track operator errors during a surgical training procedure and assess proficiency. Thus, competency assessment can be made based upon standards established by an external authority. For instance, acceptable standards of treatment expertise might require that a caregiver is able to perform a procedure correctly at 95% accuracy, as determined by training doctrine for that situation, while in other situations acceptable success levels may require only 75% success. These various standards can be incorporated into the software so that advancement to a more difficult training level is predicated upon successful completion of the lower training levels. Performance statistics can be recorded for each trainee and remain as a permanent record of achievement at various points in time. In this manner, early learning curve experience, maintenance experience, and failing performance levels can be recognized. Such records can also be accessed by secure Internet connections so that performance can be reviewed by an examiner remotely situated relative to the training exercise.

In a further aspect of the invention, the instrument tracking module follows instrument motion and is integrated with augmented reality displays of the casualty's internal anatomy. That is, a trainee can see a display of the internal region of the mannequin along with a tracked instrument. This ability to “see through” an opaque object can be referred to as augmented reality view. The augmented reality interface is totally integrated with the more general user interface and learning system of the simulator. Both components exchange the information required to provide the appropriate feedback for each scenario implemented in the system. Exemplary scenarios include simple procedures or a combination of several procedures. In each case the tracking devices and various software components are reconfigured according to the specifics of the procedure that is being performed, making the system highly flexible.

Moreover, since the steps of the training procedures have been implemented in the software system, the potential errors that could be made by the trainee are tracked in real-time, thus allowing minimal human supervision during the training. For example, the electromagnetic tracking module can determine precise instrument placement path and location of the chest dart or chest tube relative to a proper entry point and underlying anatomic structures.

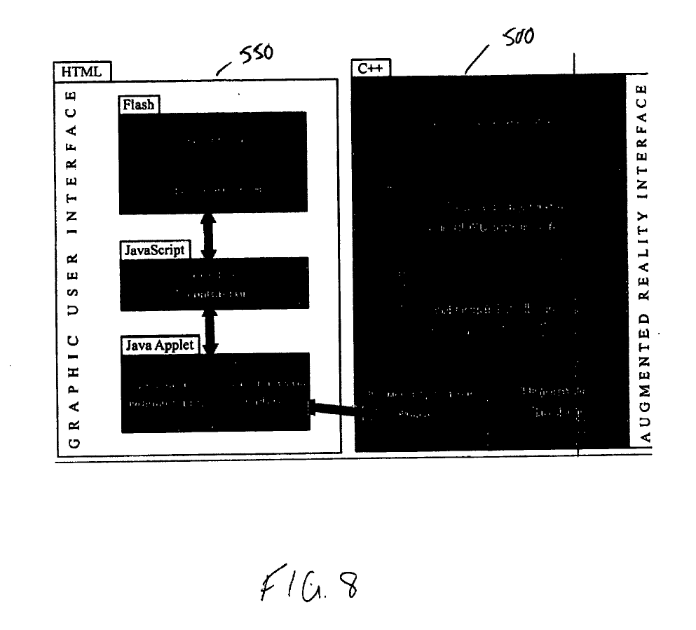

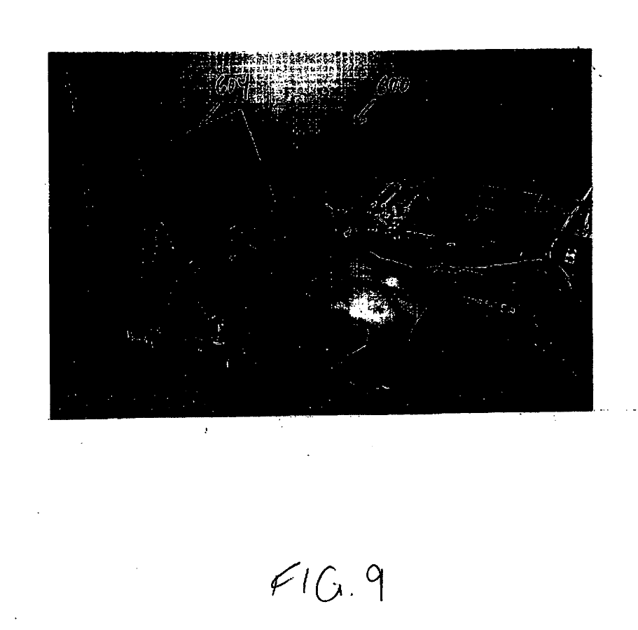

FIG. 8 shows an exemplary functional architecture for a surgical training system in accordance with the present invention. The system can include an augmented reality interface (ARI) 500 communicating with a Graphical User Interface 550, each having various modules to effect a realistic surgical training experience. In one embodiment, the ARI 500 includes an augmented reality module 502 for procedure playback capability, a graphics/sound management module 504 and an instrument tracking/collision detection module 506. The ARI 500 can further include a communication module 508 and a procedure checking module 510.

The GUI 550 can include a Flash component having an interface module 552 and action script module 554, which interacts with a Flash/Java communication module 556. The GUI 550 can further include a scenario management module 558 along with a communication module 560 for communicating with the ARI 550. In one particular embodiment, the GUI 550 components can be written in FLASH (Macromedia) and the JAVA programming language. The ARI 500 can be written in the C programming language. One of ordinary skill in the art will recognize that the system can be implemented in various hardware and software architectures using any suitable programming language without departing from the present invention.

In general, the GUI 550 is the bridge between the physical patient, as embodied by the mannequin, and the treating medical personnel. In an exemplary embodiment, the GUI 550 includes Flash action scripts 554 providing, using Macromedia Flash for example, different presentations that the user interacts with on the touch screen and various function modules 558, such as Java applets, which can be integrated with HTML.

The Flash interface 552 includes the visual information that is used as the training sessions unfold. Exemplary Flash screens include registration and identification functions, multiple diagnostic and medical choices, explanations of the procedures, indications of errors, and command screens. The screens can be displayed using a mixture of text, pictures, and Flash functionalities like animations and ActionScript code.

The Java function modules 558,560, e.g., Java applets, handle communications between the Flash interface 556 and other subsystems, such as the instrument tracking module 506 and other augmented reality visualization subsystems. In one particular embodiment, Flash FSCommands are used to communicate from the FLASH interface to the Javascript code contained in the HTML file. The Java Native Interface (JNI) then communicates with the augmented reality subsystems, which can be written in C. Java also uses multi-threading capacity to handle error tracking and success/failure reports for each user, which are used to generate individual reports on trainee performance. This performance is initiated and monitored in the FLASH user interface.

Module applets are used to control the level of training proficiency. Each training levels, e.g., seven levels, is defined as a distinct Java object, containing all the navigation and response parameters to drive the FLASH interface so that it responds to the user correctly. This architecture provides a straightforward way to adapt the training levels to the user's needs. With this arrangement, the system can also generate scenarios randomly during examination for certification of proficiency and competency.

The instrument tracking module 506 can track the position of one or more instruments at once, as well as track the movement of each instrument over a series of procedures. Unconstrained free-hand motion of the instruments during treatment of the injury can be recorded and subsequently displayed for the trainee and the trainer by the augmented reality module 502. For example, the chest dart's point of entry and final position relative to the collapsed lung beneath can be displayed on demand so that the proper technique can be learned. Similarly, the location of the syringe to administer local anesthetic and the tip of the chest tube as it enters the body and then comes to rest can be tracked. Because the system permits free-hand tracking of any instrument position, improper placement or errors are also recorded.

Referring again to FIGS. 6 and 7, the special effects module 400, in combination with the augmented reality interface 500, generates various simulated blood and air releases to provide realistic feedback during simulated surgical procedures. In normal operation, solenoids 414 for the air valves are closed, and the air pump 402 charges the air reservoir 404 to a pressure of approximately 0.3 atmospheres, for example. In one embodiment, the air reservoir 404 includes an expandable, nearly constant pressure elastic reservoir (e.g. rubber balloons) contained inside a rigid container with a volume of approximately 400 ml. The reservoir 404 provides a known volume of pressurized air, while the elastic element maintains the pressure as the air is discharged.

In normal operation, the simulated blood flows through a solenoid valve SD3 controlled by parallel port pin D3 from the blood container 408 into the measured chamber 410 by the compressed air generated by air pump 402, stored in air reservoir 404 and released to pressurize the blood container 408 through solenoid valve D5. In this state, all other valves are closed, preventing undesired fluid or air flows.

If a pneumo-thorax is treated successfully, solenoid valve SD0 opens, allowing the air charge in air reservoir 404 to be released through the chest tube. Simultaneously, valves SD5 and SD3 are closed to preserve synthetic blood and air pressure in blood container 408 and measured chamber 410. After a predetermined period, valve SD0 closes, and the valve state is returned to the “normal operation” condition described above to permit the air reservoir to recharge.

If a pure hemothorax is treated successfully, solenoid valve SD2 opens, pressurizing the measured blood chamber. Solenoid valve SD4 opens allowing the blood to be discharged through the chest tube, as the air pressure within the measured chamber causes the elastic balloon, in which the blood is stored, to collapse. Simultaneously, valves SD5 and SD3 are closed to prevent loss of synthetic blood back into the blood container 408. Once the balloon has completely collapsed, blood flow ceases and after a predetermined period, the valves are reset to the “normal operation” condition. In this condition, the measured chamber 410 is depressurized via valve SD2, which is a 3-way valve, with an exhaust port to release pressure from the “outlet” side, when it is in the “closed” state. (When in the “open” state, the exhaust port is closed). This depressurization is necessary to permit the internal balloon in chamber 410 to refill.

If a hemo-pneumothorax is treated successfully, valve SD1 opens, injecting air into the synthetic blood-filled balloon within measured chamber 410. Valve SD4 simultaneously opens, releasing the synthetic blood from the balloon and allowing it to be discharged through the chest tube. Simultaneously, valves SD5 and SD3 are closed to prevent loss of synthetic blood back into the blood container 408. Once the air charge from air reservoir 404 has been expended, the elasticity of the balloon within measured chamber 410 causes the majority of the remaining air to be expelled from the chamber and through the chest tube. After a predetermined period, the valves are reset to the “normal operation” condition. The measured chamber 410 is depressurized via valve D2, and the internal balloon in chamber 410 refills.

Alternatively, the system can include an on-demand type of air pump with a large flow capacity, so as to eliminate the need for the continuously running the air pump and the constant pressure air reservoir, as the on-demand pump would sense a drop in pressure below the desired valve and then activate to maintain pressure. In the exemplary embodiment, the valves as shown have a Cv value (a rating of flow capacity) of at least 0.61 for valves that pass only air, and 1.7 for valves that pass fluid. Other ratings may be used provided that they do not have significantly higher flow resistances, which would reduce the fluid output through the chest tube.

In one particular embodiment, the synthetic blood is a mixture of 4 to 5 parts water to each part red tempera paint (Sargent Art, Inc., Hazleton, Pa., 18201, part number 22-4220). Other substitutes with similar viscosity, color and opacity may be employed.

In a further aspect of the invention, after a user has completed the surgical procedure, the system displays an augmented reality playback animation which literally replays the user's actions on the mannequin. Since certain instruments can be tracked to determine illegal collisions the position and orientation of these sensors can be saved to the controlling computer at a regular interval while the user is working on the mannequin. This recorded sensor log file can then be used to drive a virtual 3D scene to permit the user to see his or her actions played back in front of them. The augmented reality module starts, for example, by displaying a corresponding virtual mannequin on a litter without a shirt or jacket and without arms. The sensor samples are then read incrementally from the log file and used to position a corresponding surgical instrument model within the computer scene. Thus, the virtual instrument follows the same user's path that they performed on the mannequin. When the driven instrument model penetrates the skin model, the skin responds by fading away to display a series of internal anatomy models consisting, for example, of the rib cage, lungs, mediastinum, and diaphragm.

These same anatomical models were used in the collision detection process, as described above. As the playback continues with this internal view, users can now clearly see the instrument's tip in relationship to the internal anatomy. If the user hit an internal organ previously on the mannequin, the corresponding playback will clearly demonstrate the collision since the instrument model will visually penetrate one of the organ models.

The augmented playback feature provides the user with ‘x-ray vision’ into their actions within the mannequin which they cannot see in real life. It reinforces the spatial relationships which are critical for a successful treatment. Subjects can clearly visually see errors that the system flagged during their session or how close they came to an error. It also gives a supervisor a way to review and critique a user's performance.

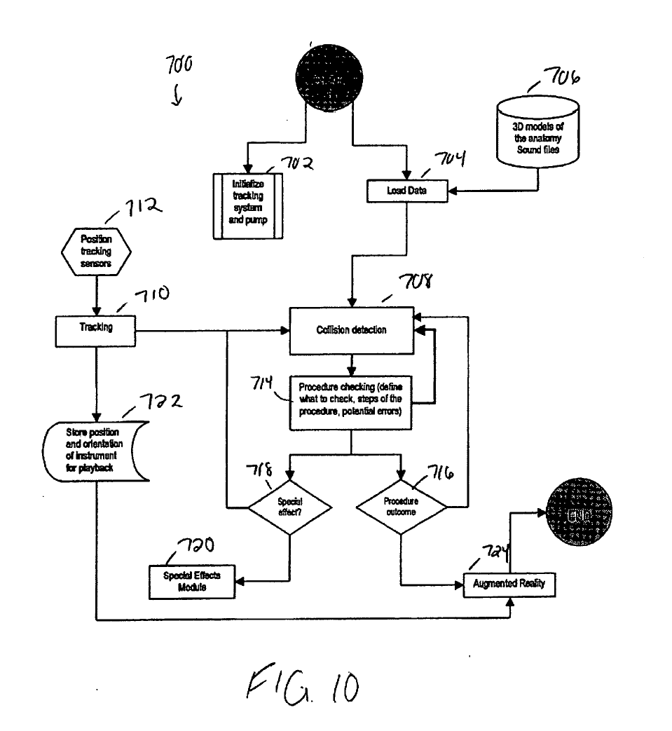

FIG. 9 shows an exemplary litter 600 having a support structure/mounting assembly 602 for securing the display screen 604 to convey visual information and a text interface to the trainee. The mounting assembly 602 can be easily attached to and removed from the litter 600 for ease of assembling the system. The preferred embodiment includes a means to pivot the monitor 604 to the left and right sides of the litter, for the convenience of displaying information whichever side treatment is being performed on. In one particular embodiment, the support structure includes PVC tubing, pipe fittings, four hose clamps and a rail fitting to support the monitor. A custom-made aluminum bracket holds the monitor at a convenient viewing angle, and permits attachment to the rail fitting. It is understood that a wide range of alternative embodiments will be readily apparent to one of ordinary skill in the art.

As described above, the visual interface 604 can provide visual cues, instructional elements for proper dart placement and chest tube insertion, and audible cues via an integrated speaker when tension pneumothorax is relieved. Synthetic voice commands also guide the trainee in proper timing of therapeutic maneuvers.

When combined with the augmented reality display, the ability to track errors as well as correct technique provides the system a degree of “smart mannequin” capability. For example, if a trainee punctures the lung or liver on early training sessions but learns the proper technique through rehearsal and repetition, improvement and advancement to more sophisticated levels of training can occur. Conversely, progression to more difficult treatment methods is not permitted until simpler techniques are successfully completed. Criteria for success can be established by an outside authority, whether an examining board or a course certification requirement, and the software can be programmed to reflect new or changing requirements as required by new doctrine or various corps requirements.

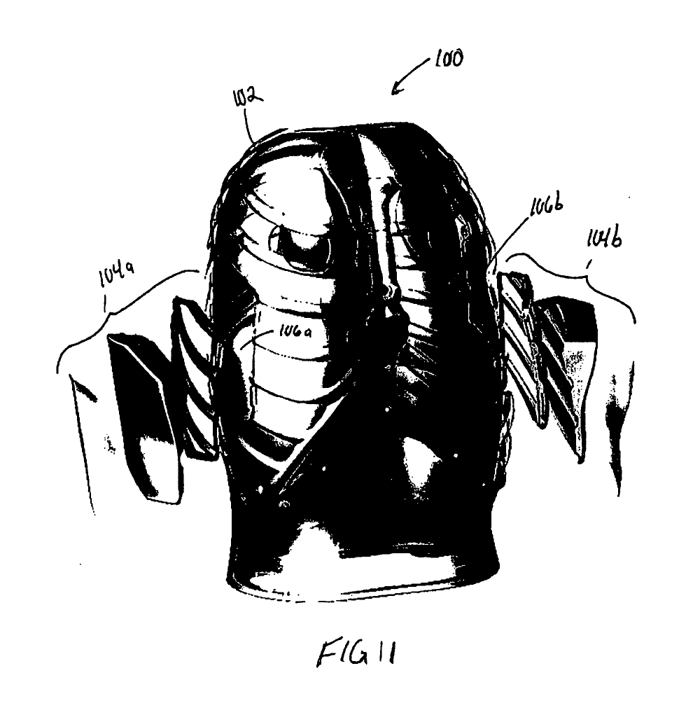

FIG. 10 shows a top level interaction diagram for an exemplary medical training system 700 in accordance with the present invention. Initially, the system is initialized 702 and data for a selected procedure is loaded 704 from a database 706. For the procedure, the collision detection module 708 receives information from the database 706, the instrument tracking module 710, which receives instrument location information from the tracking sensors 712, and a procedure checking module 714. In an exemplary embodiment, the procedure checking module defines what information is to be checked, e.g., instrument locations, the steps for the selected procedure, as well as errors, potential errors and close calls.

During the training procedure, the collision detection module 708 and the procedure checking module 714 combine to determine the procedure outcome 716 and the whether a special effect 718, e.g., simulated blood flow, should be activated by the special effects module 720. Over the course of the procedure, the instrument location can be tracked and stored 722 by the system for later playback by the augmented reality module 724, which can show instrument movement in relation to the mannequin as described above.

In another aspect of the invention, the surgical training system provides a number of apertures in the mannequin in which particular anatomical sections referred to as portals, can be interchanged. The replaceable portals can be chosen in areas which are altered by a particular surgical procedure. As a result, portal sections may need to be replaced after each training session. As described below, the portals can be made with high grade materials resulting in a very realistic “look” and “feel” compared to a real human subject. It is understood that the portal described below can form a part of the systems described above.

Before further describing this aspect of the present invention, some introductory concepts and terminology are explained. As used herein, the term “portal” refers to a device having predetermined geometries and anatomically analogous characteristics that supplement the surgical training mechanism. That is, in an exemplary embodiment, the portal is constructed such that a particular surgical training procedure using the portal “feels” like the corresponding anatomical structure on a patient. And as described above, the portal can be fabricated based upon 3D models derived from medical images of a human subject.

Also, it should be appreciated that, in an effort to promote clarity, reference is sometimes made herein to portals being located in certain positions on a torso or mannequin. Such references should not be taken as limiting the scope of the present invention to construction/use of portals in only those locations on a torso. Rather, the portals of the present invention can be used in any location on a torso. It should also be appreciated that in some applications the portal can be used without a torso. In addition, while the invention is described in conjunction with exemplary surgical procedures, further procedures and corresponding portals, will be readily apparent to one of ordinary skill in the art and within the scope of the present invention.

It should be further appreciated that the term “torso” generally refers to a portion of a human body extending from the junction of the neck and chest to the armpits to the bottom of the ribcage or waist. As used herein, the term torso should be broadly construed to include a full body torso, which can include a head, arms, legs, and portions thereof, as well as any portion of a full body torso.

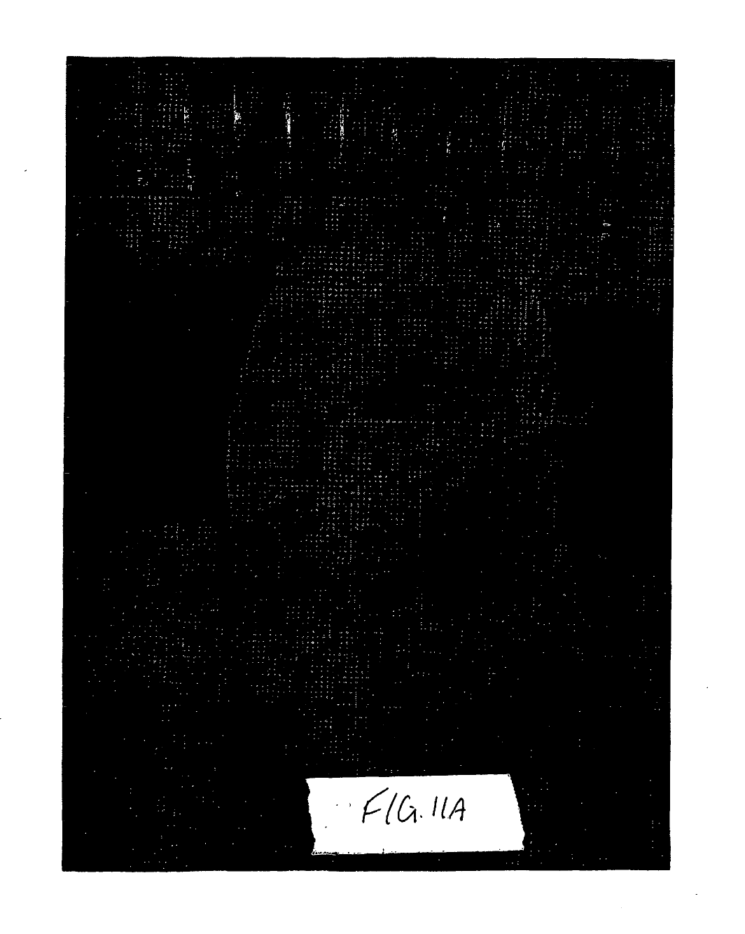

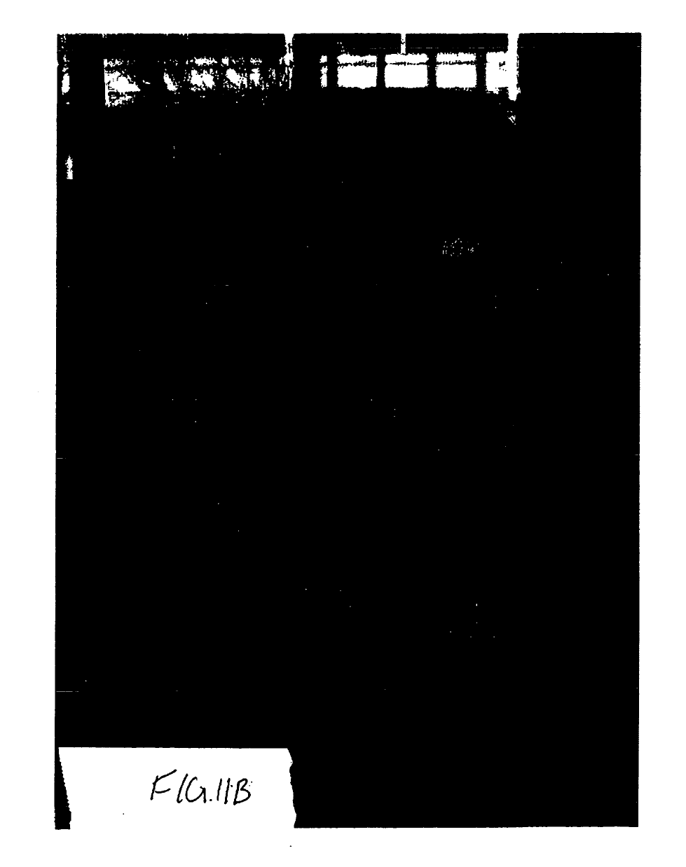

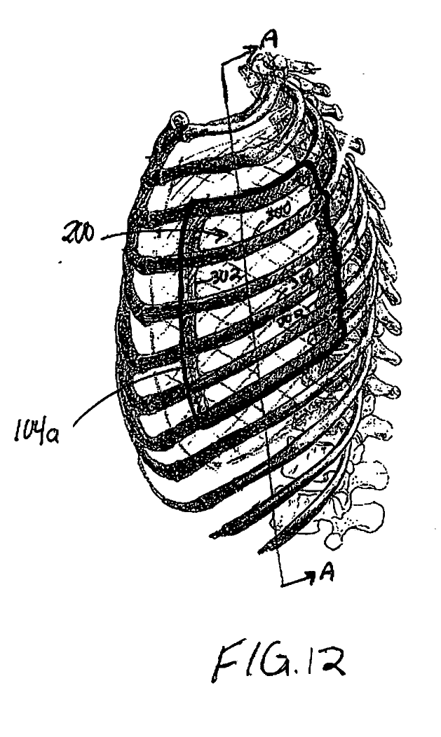

FIG. 11 shows a surgical training system 100 including a torso shell 102 having left and right lateral chest portals 104a,b, which are shown in an exploded view, providing anatomically analogous features in accordance with the present invention. The torso shell 102 provides a relatively rigid structure with left and right apertures 106a,b into which the respective portals 104a,b are removably insertable.

In general, the lateral chest portals 104 provide a realistic artificial interface of a portion of the right and/or left lateral chest wall for training physicians, students, medical technicians, nurses, paramedical personnel, and military trainees in various surgical procedures, such as inserting a chest tube for management of chest trauma. It is understood that the size of the chest tube can vary. The portal is comprised of anatomically analogous layers of material fabricated to reproduce the feeling of incising and puncturing the skin, subcutaneous fat, intercostal muscle, ribs, and parietal pleural surface during blunt and sharp dissection of a patient's chest and insertion of a chest tube. For example, the inventive portals can be used to train/teach techniques for placement of a standard 36 Fr chest tube for treatment of pneumothorax and hemothorax, and placement of a 10 Fr chest “dart” for tension pneumothorax.

FIG. 11A shows a further view of the torso shell 102 to which the left lateral chest portal 104a is secured. FIG. 11B shows the torso shell 102 covered by a flexible outer layer 108 for a more realistic appearance. The flexible outer layer 108 can be comprised of various materials to provide a life-like appearance and feel. In one particular embodiment, the outer layer 108 is provided as Chest Drain Epidermis version 1.1, by Limbs & Things of Bristol, England. The torso shell 102 can be formed from a variety of suitable rigid and semi-rigid materials. In one particular embodiment, the shell 102 is formed from a plastic material, such as polyurethane. The torso shell should be sufficiently rigid to resist deformation during forceful inward pushing of the instruments and tube during the procedure and accurately represent the underlying anatomic structures such as the ribs.

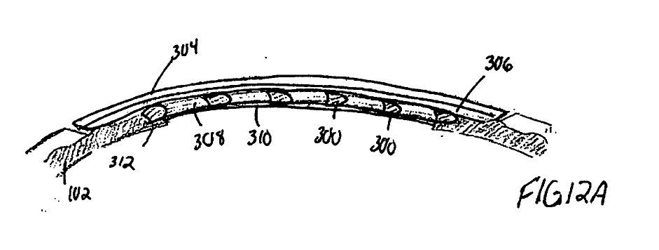

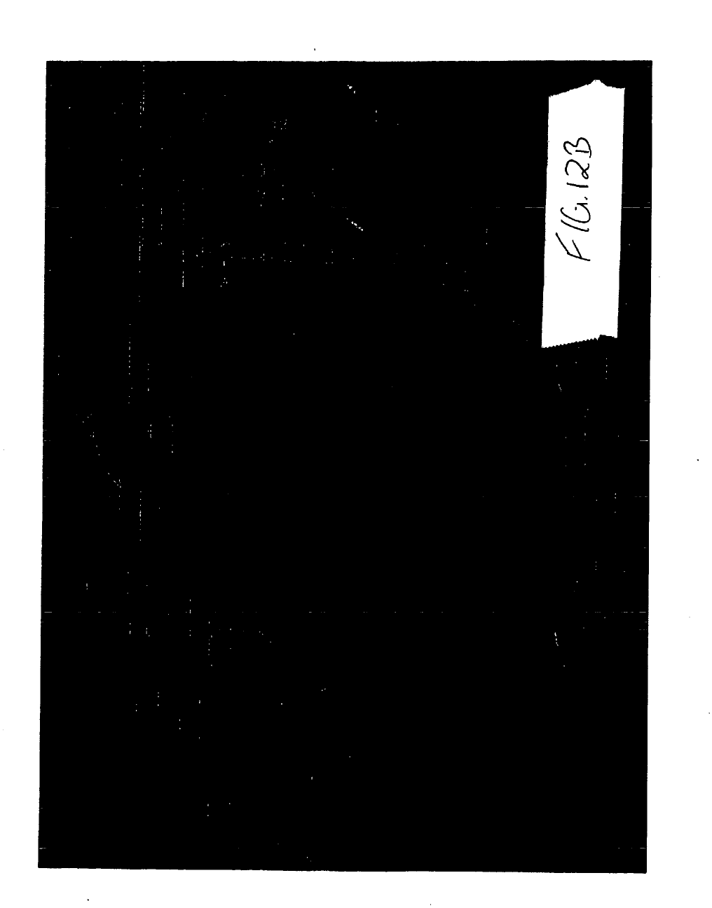

FIG. 12 shows a skeletal view of an anatomical region 200 into which a lateral chest portal, such as the lateral chest portals 104a,b of FIG. 11, can be removably inserted. For clarity, the portal is shown with ribs 300 and a frame 302 but without certain anatomic layers, which are shown in FIG. 12A. It is understood that the inventive portal can have a wide range of geometries based upon a particular application/surgical training procedure. For example, the number and location of ribs emulated by the portal can vary. The particular anatomic location represented by a portal can vary depending on the procedure and the number of layers required for realistic portrayal of the area of interest. In one particular embodiment, the lateral chest portal 104 comprises an anatomical chest region corresponding to a portion of the fourth to the eighth ribs of the lateral mid-axillary section of an adult male torso.

FIG. 12A shows a cross-sectional view of the lateral chest portal 104 of FIG. 12 along lines A-A including anatomically analogous layers, some of which are not shown in FIG. 12. The lateral chest portal 100 includes a “skin” layer 304 covering a “subcutaneous fat” layer 306 disposed over “intercostal muscle” 308, which surrounds the “ribs” 300. In one embodiment, the ribs 300 are embedded in the intercostals muscle 308. It is understood that the majority of the intercostal muscle material 308 will be between adjacent ribs 300 and that the extent to which the ribs are embedded can vary to meet the needs of a particular application. Alternatively, the intercostal muscle material 308 does not surround the ribs 300, but rather, is located between ribs.

The portal can further include a “parietal pleura” layer 310 on the opposing (inner anatomic) side of the intercostal muscle 308 covering the ribs 300. It is understood that each layer corresponds to its anatomical equivalent. The lateral chest portal 104 further includes the frame 302 (FIG. 12) from which the ribs 300 extend to provide structural integrity to the portal.

The torso shell 102 can include a shelf structure 312 to support the lateral chest portal 104 on the torso along with an engagement mechanism for retaining the portal in place during procedures. It will be readily apparent to one of ordinary skill in the art that a wide range of alternative engagement mechanisms can be used without departing from the present invention.

FIG. 12B shows an exemplary engagement mechanism 400 includes screw-mounted tabs 402, which turn on the axis of the screw 404 to either cover and hold a small region of the portal, or rotate out of the way of the portal to permit removal.

The lateral chest portal can comprise various materials that are suitable for providing realistic haptic feedback. The ribs/frame can be formed from molded polyurethane in a shape that allows the portal to rest on the corresponding aperture in the torso shell. The ribs/frame should be sufficiently rigid so as to handle the forces expected during the particular procedure. Over and around the ribs is poured a mold of the intercostal muscle material for the appropriate rib segments. In one particular embodiment, the intercostal muscle material is provided as Chest Drain Muscle version 1.13 by Limbs and Things, which is cast onto the ribs. The intercostal muscle material is overlaid with a fat-like material corresponding to the proper thickness for the anatomic region in the mid thoracic mid axillary line, with thicker fat at the uppermost aspect and thinner fat at the inferior margin. Suitable fat materials, such as methacrylate-based polymer blends, are well known to one of ordinary skill in the art. In an exemplary embodiment, the fat material is provided as Chest Drain Fat version 1.9 by Limbs and Things. The fat layer is overlaid with a skin material, which is selected to have characteristics that permit realistic cutting with a scalpel and suturing characteristics when the material is sewn, i.e., the material exhibits characteristics similar to living human skin, retracts and maintains adherence to the underlying layer when dissected and can be re-apposed through the use of surgical repair materials, such as suture or staples or other liquids or solids. The portal is completed with a tightly adherent innermost layer of fabric/latex sandwich or other materials that replicate the material haptic sensations of a resistant layer that simulates the properties of the parietal pleura. In one embodiment, the parietal pleura is provided as Chest Drain Pleura version 1.2 by Limbs and Things.

The portal materials together provide the sensations that would be felt during sharp and blunt dissection through the several layers of the chest wall. For example, the portal permits realistic palpation of the underlying ribs, skin incision with a scalpel, dissection with a finger or instrument, and chest tube or chest dart insertion. It is understood that the materials should maximize re-usability of the portal to the extent reasonably possible.

Referring now to FIGS. 3 and 3A, in another aspect of the invention, an anterior chest portal 400 (shown as left and right anterior chest portals 400a,b) is provided for tension pneumothorax training. The torso shell 402 includes left and right apertures 404a,b corresponding to the left and right anterior chest portals 400a,b. It is understood that under normal training conditions, a flexible outer layer, such as the outer layer 108 of FIG. 11B, will cover the torso shell 402.

In one embodiment, the anterior chest portal 400 is designed as part of an adult male torso for providing a realistic feel of puncturing the anterior chest wall during insertion of a large gauge (e.g., 10 gauge) chest dart. The anterior chest portal 400 facilitates learning of the proper forces and typical resistance during safe insertion of a chest dart between the uppermost two ribs (number 2 and 3 ribs) in a simulated trauma

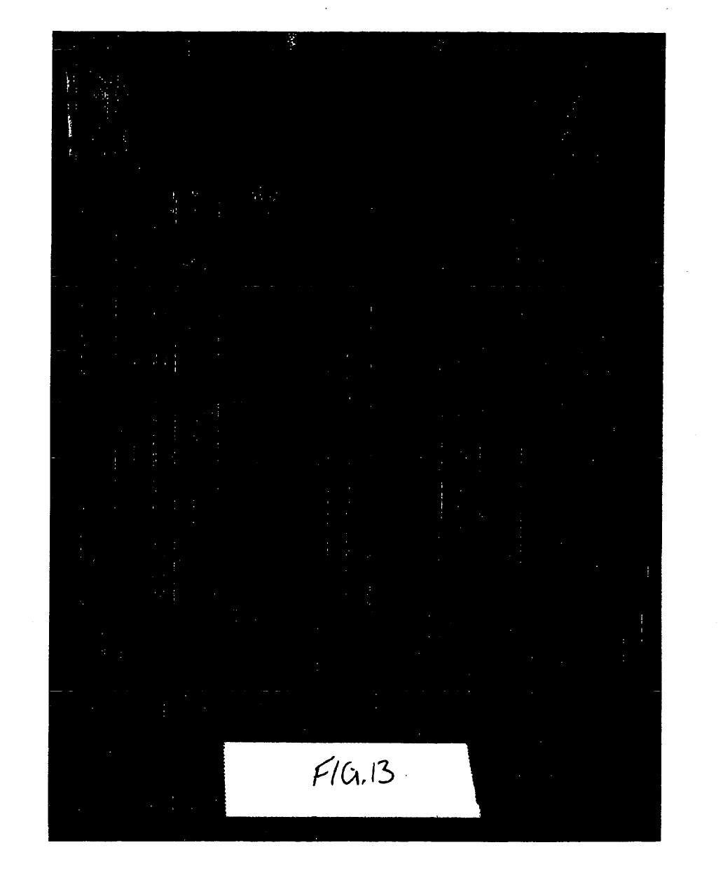

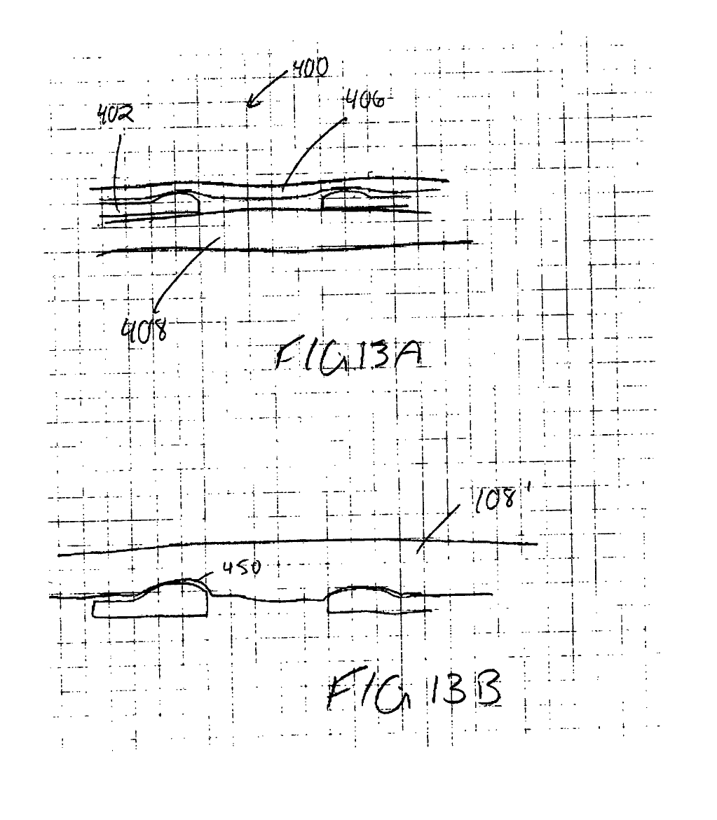

As best shown in FIG. 13A, the anterior chest portal 400 includes a skin surface layer 406, which can be provided as part of the flexible outer layer 108 (FIG. 11B), disposed over a subcutaneous layer 408 of a uniform cross-linked latex foam material that provides resistance similar to the pectoral muscle. In an exemplary embodiment, the anterior chest portal 400 can be punctured many times without breaking down.

Alternatively, as shown in FIG. 13B, the outer layer 108′ can comprise an integral layer to provide the desired haptic feedback for the portal. The layer 108′ can form fit over the torso shell with indentations 450 corresponding with the palpable rib forms of the shell. The outer layer 108′ provides the appropriate resistance to puncture and the like.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1-3. (Cancelled).

4. A portal comprising:
a support structure;
a plurality of members corresponding to ribs secured to the support structure, the plurality of members defining an exterior side and an interior side;
a first material secured to the plurality of members, the first material corresponding to intercostal muscle;
a first layer adjacent to the first material and disposed on the interior side of the plurality of members, the first layer corresponding to pleura layer;
a second layer adjacent the first material and disposed on the exterior side of the plurality of members, the second layer corresponding to subcutaneous fat; and
a third layer adjacent the second layer, the third layer corresponding to skin.

5. The portal according to claim 4, wherein the plurality of members are embedded in the first material.

6. The portal according to claim 4, wherein the portal is adapted to provide haptic feedback during insertion of a tubular instrument into the portal that emulates insertion of the tube into a human.

7. The portal according to claim 6, wherein the tubular instrument corresponds to about a 36 French chest tube.

8. The portal according to claim 4, further including a torso shell to which the portal can be secured.

9. The portal according to claim 8, further including an outer layer disposed over the torso shell for providing a realistic appearance.

10. The portal according to claim 8, wherein the torso shell includes an aperture corresponding to the portal.

11. The portal according to claim 10, further including an engagement mechanism for removably securing the portal to the torso shell.

12. The portal according to claim 4, wherein the portal is derived from medical imaging of a human.

13. A surgical training system, comprising:
a torso shell having a first aperture in a location corresponding to a lateral chest wall;
a lateral chest portal that is removably insertable into the first aperture in the torso shell, the lateral chest portal including
a support structure;
a plurality of members corresponding to ribs secured to the support structure, the plurality of members defining an exterior side and an interior side;
a first material secured to the plurality of members, the first material corresponding to intercostal muscle;
a first layer adjacent to the first material and disposed on the interior side of the plurality of members, the first layer corresponding to pleura layer;
a second layer adjacent the first material and disposed on the exterior side of the plurality of members, the second layer corresponding to subcutaneous fat; and
a third layer adjacent the second layer, the third layer corresponding to skin.

14. The system according to claim 13, wherein the plurality of members are embedded in the first material.

15. The system according to claim 13, wherein the lateral chest portal is adapted to provide realistic haptic feedback during insertion of an elongate instrument into/through the portal that emulates insertion of the tube into a human.

16. The system according to claim 13, further including a flexible outer layer fitting over the torso shell.

17. The system according to claim 13, further including an engagement mechanism for removably securing the portal to the torso shell.

18. The system according to claim 13, wherein the portal support structure includes a support region for complementary coupling about the first aperture in the torso shell.

19. The system according to claim 13, wherein the portal has anatomical characteristics derived from medical imaging of a human.

20. A method of surgical training, comprising:
inserting a tubular instrument into a torso through a portal with realistic haptic feedback, the portal having
a support structure;
a plurality of members corresponding to ribs secured to the support structure, the plurality of members defining an exterior side and an interior side;
a first material secured to the plurality of members, the first material corresponding to intercostal muscle;
a first layer adjacent to the first material and disposed on the interior side of the plurality of members, the first layer corresponding to pleura layer;
a second layer adjacent the first material and disposed on the exterior side of the plurality of members, the second layer corresponding to subcutaneous fat; and
a third layer adjacent the second layer, the third layer corresponding to skin.

21. A method of forming a portal for simulating a surgical procedure, comprising:
medically imaging a human;
generating a dataset from the medical images;
segmenting the dataset to identify the anatomical region corresponding to the portal;
generating a three-dimensional design file for the portal from the segmented dataset; and
fabricating the portal from the design file.
